(19) United States
(12) Patent Application Publication
(10) Pub. No.: US 2016/0310251 A1
(43) Pub. Date: Oct. 27, 2016

(54) USER CONFORMABLE INCONTINENCE CLAMP

(71) Applicant: Erich Rolko, Agoura Hills, CA (US)

(72) Inventor: Erich Rolko, Agoura Hills, CA (US)

(21) Appl. No.: 15/135,065

(22) Filed: Apr. 21, 2016

Related U.S. Application Data
(60) Provisional application No. 62/150,485, filed on Apr. 21, 2015.

(51) Int. Cl. A61F 2/00; A61B 17/122

(52) U.S. Cl. CPC A61F 2/0054; A61B 17/122; A61F 2220/0091; A61F 2250/0078; A61F 2250/0004

(57) ABSTRACT

A user conformable incontinence clamp includes two plastically deformable wires, a first end of each wire elastically connected at a flexible hinge. A second end of each wire includes a selective attachment mechanism that can be connected together to form a continuous loop. The user conformable incontinence clamp further includes a padded tube that provides adequate force distribution for user comfort. To use the user conformable incontinence clamp, a user may open the clamp, position his penis within the clamp, close the clamp around his penis, and then plastically compress the clamp against his penis to provide a desired level of incontinence protection.

### CROSS-REFERENCE TO RELATED APPLICATIONS

The present application claims benefit of priority to U.S. Provisional Patent Application No. 62/150,485, entitled “User Conformable Incontinence Clamp,” and filed on Apr. 21, 2015, which is specifically incorporated by reference herein for all that it discloses or teaches.

### BACKGROUND

Urinary incontinency, incontinence, or involuntary urination (collectively “UI”) is the involuntary leakage of urine from an individual's urinary tract. UI is a common and distressing problem, which may have a profound impact on the individual's quality of life. Several types of devices are available to individuals who experience UI to help them manage their individual situation. Due to the external nature of the male urethra and internal nature of the female urethra, incontinence devices are typically sex specific, with a majority of devices directed to the male penis due to ease of physical access. Example incontinence devices include collecting systems, absorbent products, fixer-occluder devices, indwelling catheters, and intermittent catheters.

Fixer-occluder devices (also referred to herein as “incontinence clamps”) are attached to the penis, compressing the urethra and surrounding penis to reduce or prevent the flow of urine there through. Existing incontinence clamps tend to be bulky, uncomfortable, rigid, and non-adjustable (or with limited adjustment). As a result, existing incontinence clamps tend to be effective only for light or moderate incontinence.

### SUMMARY

Implementations described and claimed herein address the foregoing problems by providing a user conformable incontinence clamp comprising: a first wire including a first selective attachment mechanism on a first end of the first wire; a second wire including a second selective attachment mechanism on a first end of the second wire; a flexible hinge connecting a second end of the first wire to a second end of the second wire; and a padded tube encompassing a majority of the clamp, wherein the first selective attachment mechanism is configured to selectively attach to the second selective attachment mechanism to close the clamp around a user's penis, and wherein the first wire and the second wire are selectively conformable to the user's penis.

Implementations described and claimed herein address the foregoing problems by further providing a method of using a user conformable incontinence clamp comprising: positioning the user conformable incontinence clamp at a desired location on a user's penis; closing the clamp around the user's penis; and compressing the clamp to conform to a desired shape and level of compression on the user's penis.

Other implementations are also described and recited herein.

### BRIEF DESCRIPTION OF THE DRAWINGS

FIG. 1A illustrates an example user conformable incontinence clamp in an unclamped position.

FIG. 1B illustrates the example user conformable incontinence clamp of FIG. 1A in a partially clamped position.

FIG. 1C illustrates the example user conformable incontinence clamp of FIGS. 1A and 1B in a fully clamped position.

FIG. 2 is a cross-section of an example user conformable incontinence clamp in an open position.

FIG. 3 is a cross-section of an example user conformable incontinence clamp in a closed position.

FIG. 4 illustrates example operations for using a user conformable incontinence clamp.

### DETAILED DESCRIPTIONS

User conformable incontinence clamps are disclosed herein that may address some or all of the aforementioned shortcomings identified in prior art incontinence clamps.

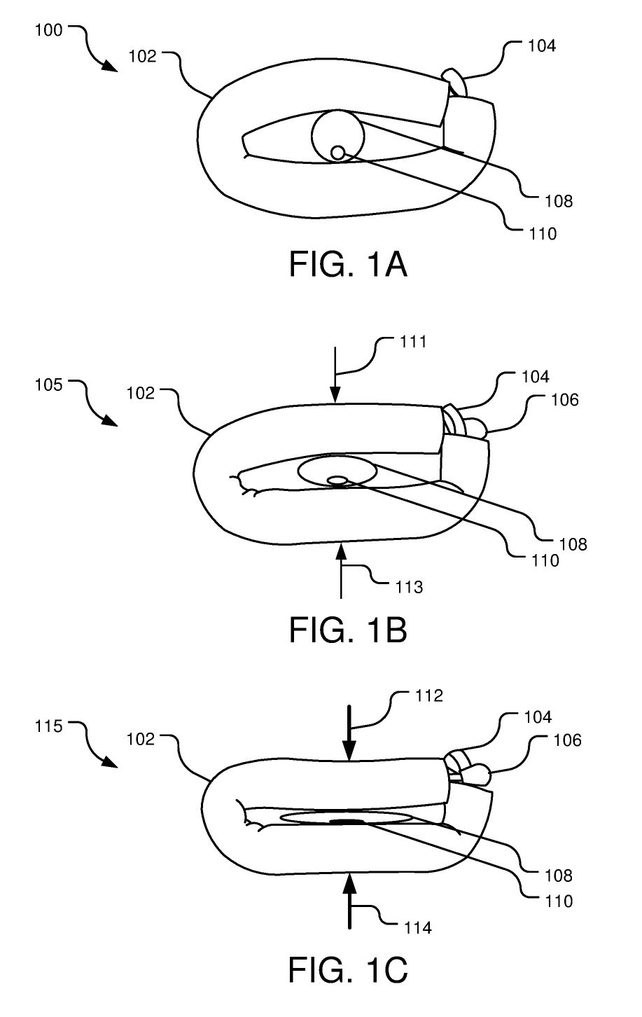

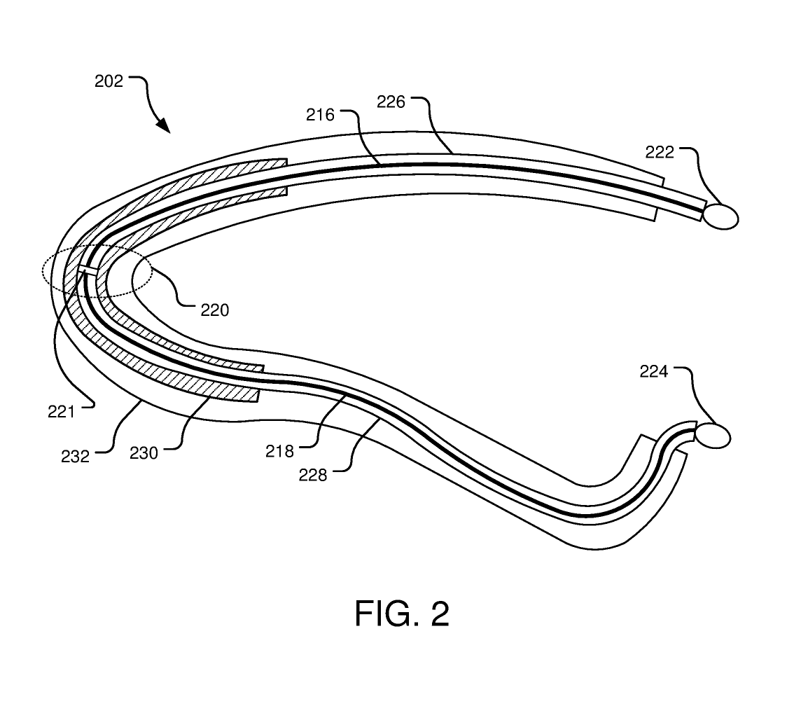

FIG. 1A illustrates an example user conformable incontinence clamp 102 in an unclamped position 100. The clamp 102 forms a loop of plastically deformable material that is selectively closed by attaching a first hook 104 to a second hook (not shown) of the clamp 102. In other implementations, the clamp 102 forms a permanently closed loop with no hooks or other fasteners. Structural details of the clamp 102 are discussed below with reference to FIG. 2.

In operation, the clamp 102 is closed around a penis 108 by extending the penis 108 through the loop and attaching the first hook 104 to the second hook. A schematic cross-section of the penis 108 is shown, which includes a urethra 110. The urethra 110 is to be compressively closed by action of the clamp 102 as discussed with reference to FIGS. 1B and 1C below.

FIG. 1B illustrates the example user conformable incontinence clamp 102 of FIG. 1A in a partially clamped position 105. As discussed with regard to FIG. 1A, the clamp 102 forms a loop of plastically deformable material that is selectively closed around the penis 108 by attaching a first hook 104 to a second hook 106 of the clamp 102. In other implementations, the clamp 102 forms a permanently closed loop with no hooks or other fasteners.

Opposing compressive forces 111, 113 are applied to the clamp 102, which exceed the yield strength of the underlying clamp material. As a result, the clamp 102 is plastically deformed in a manner that compresses the penis 108 and the urethra 110 as shown. Compression of the surrounding penis 102 compressively pinches the urethra 110, thereby reducing the cross-sectional area of the urethra 110. A rate at which urine may flow through the urethra 110 is proportionally reduced as the cross-sectional area is reduced. In various implementations, a user's thumbs and fingers apply the compressive forces 111, 113 and may vary the location, magnitude, and direction of the compressive forces 111, 113 from that shown in FIG. 1B. For example, the user may pinch the clamp 102 to provide the compressive forces 112, 114.

FIG. 1C illustrates the example user conformable incontinence clamp 102 of FIGS. 1A and 1B in a fully clamped position 115. As discussed with regard to FIGS. 1A and 1B, the clamp 102 forms a loop of plastically deformable material that is selectively closed around the penis 108 by attaching a first hook 104 to a second hook 106 of the clamp 102. In other implementations, the clamp 102 forms a permanently closed loop with no hooks or other fasteners.

Increasing opposing compressive forces 112, 114 are applied to the clamp 102 to provide additional plastic deformation of the clamp 102 and overcome any resistance to compression provided by the compressed penis 108 until the clamp 102 reaches the fully clamped position 115. When the clamp 102 reaches the fully clamped position 115, the user releases the compressive forces 112, 114 and the rigidity of the clamp 102 reduces or prevents deformation of the clamp 102 that would release the compressed penis 108.

In the fully clamped state, the urethra 110 is compressively pinched closed, thereby reducing the cross-sectional area of the urethra 110 to zero or near zero. Little to no urine is permitted to flow through the urethra 110 in the fully closed clamped position 115.

In various implementations, the opposing compressive forces 112, 114 of FIGS. 1B and 1C may be varied to provide a desired shape to the clamp 102 for performance and/or comfort purposes. For example, since the urethra 110 lies near the bottom of the penis 108, the user may conform a top of the clamp 102 to have a concave surface to cradle a top surface of the penis 108 and/or a convex surface to provide additional pressure on a bottom surface of the penis 108 directed specifically at the region of the penis 108 where the urethra 110 is primarily located.

Further, the opposing compressive forces 111, 113 of FIG. 1B and increasing compressive forces 112, 114 of FIG. 1C used to create the fully clamped position 115 may be varied depending on the user's UI situation, intended activity, and/or duration of use of the clamp 102. For example, the user may provide a relatively lightly clamped position if the user has a relatively minor UI situation, the user intends to be relatively inactive while using the clamp 102, and/or the user intends to wear the clamp 102 for a relatively short period of time. Conversely, the user may provide a relatively strongly clamped position if the user has a relatively significant UI situation, the user intends to be relatively active while using the clamp 102, and/or the user intends to wear the clamp 102 for a relatively long period of time.

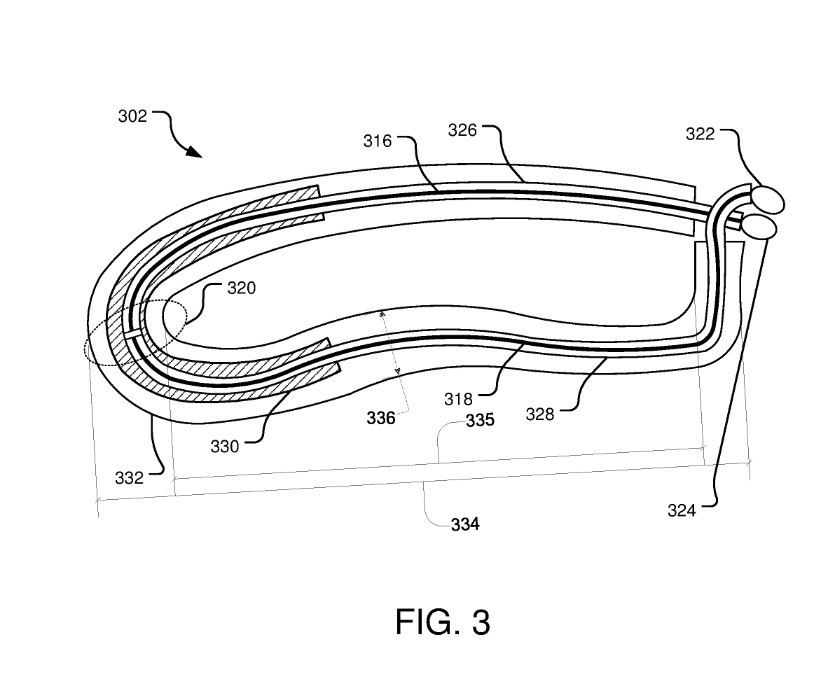

FIG. 2 is a cross-section of an example user conformable incontinence clamp 202 in an open (or unclamped) position. The clamp 202 includes solid wire cores 216, 218 that generally run through the center of the clamp 202. More specifically, the wire core 216 is separated from the wire core 218 at bending node 220 of the clamp 202. As a result, a user may selectively deform the clamp 202 at the bending node 220 without substantially deforming the wire cores 216, 218. In various implementations, the bending node 220 is elastically deformed when transitioning from the open position to a closed position (see e.g., FIG. 3), and vice versa. The wire cores 216, 218 are plastically deformed to achieve a desired contour for clamping on the user's penis.

Since the clamp 202 is primarily deformed at the bending node 220 when transitioning from the open position to the closed position, and vice versa, the wire cores 216, 218 can maintain their respective shapes even when the clamp 202 is opened and closed. This may reduce the opportunity for metal fatigue on the wire cores 216, 218 during repeated use of the clamp 202 and preserve user-selected shapes of the wire cores 216, 218 as the clamp 202 is opened and closed. Coated ends 222, 224 of the wire cores 216, 218 opposing the bending node 220 form structures for the user to physically manipulate the wire cores 216, 218, prevent the user from being poked with otherwise potentially sharp wire ends, and aid the wire cores 216, 218 selectively attaching together (see e.g., FIG. 3). In various implementations, the coated ends 222, 224 may be epoxy, plastic, or rubber coated to eliminate potentially sharp wire ends and/or aid the coated ends 222, 224 engaging together as hooks. In other implementations, the coated ends 222, 224 are replaced with hook-shaped or flattened ends that selectively attach to one another.

In various implementations, the wire cores 216, 218 are a solid metallic wires that are sufficiently flexible to be user conformable, but also sufficiently rigid to hold a selected shape in compression on the user's penis. More specifically, the wire cores 216, 218 may be made of platinum, silver, iron, copper, aluminum, gold, and various alloys thereof (e.g., brass, bronze, steel). The wire cores 216, 218 may also have any cross-sectional shape (e.g., circular, rectangular, etc.) and have a variety of gauges depending on material selection and desired rigidity of the wire cores 216, 218 (e.g., a copper alloy wire may be 10-14 gauge or approximately 12 gauge).

Wire core 216 is covered in insulation 226 and wire core 218 is covered in insulation 228. In various implementations, the insulation sections 226, 228 provide corrosion resistance to the wire cores 216, 218 and may be bonded to the wire cores 216, 218 such that the wire core/insulation combinations each deform as a single unit. In addition, an overall increased diameter and generally softer outer insulating material of the wire core/insulation combinations as compared to the wire cores 216, 218 alone may reduce or prevent the wire cores 216, 218 from cutting or otherwise damaging adjacent subsequent layers of the clamp 202 discussed below.

In various implementations, the insulation sections 226, 228 continuously wrap around the wire cores 216, 218, respectively. More specifically the insulation sections 226, 228 may be made of plastic, rubber-like polymers, and/or varnishes that cover or coat their respective wire cores 216, 218. The insulation sections 226, 228 may have any desired radial thickness depending on the desired protection afforded to the subsequent layers of the clamp 202. In some implementations, the wire core/insulation combinations are created from commercially available insulated electrical wire.

The wire core/insulation combinations are partially covered with a flexible tube 230, at least covering the bending node 220, creating a flexible hinge at the bending node 220. More specifically, axially aligned ends of each wire core/insulation combination meet at the bending node 220 and are covered by the flexible tube 230 and the flexible tube 230 extends a distance along each wire core/ insulation combination beyond the bending node 220. In various implementations, a small gap 221 is provided between the axially aligned ends of each wire core/insulation combination within the flexible tube 230 to prevent contact between the wire core/insulation combination as the flexible tube 230 is elastically deformed. As a result, the wire core/insulation combinations are attached together via the flexible tube 230 and the flexible tube 230 allows the clamp 202 to bend at the bending node 220 without substantial deformation of the individual wire core/insulation combinations. In various implementations, the wire core/insulation combinations are slip-fit, glued, welded, or otherwise securely attached to the flexible tube 230 so that the clamp 202 may be bent at the bending node 220 repeatedly without the connections between the wire core/insulation combinations and the flexible tube 230 slipping or breaking.

The flexible tube 230 is resilient, enabling the clamp 202 to readily bend at the bending node 220 repeatedly without failure. More specifically, the flexible tube 230 may be made of rubber (natural or synthetic) or plastic material. The flexible tube 230 extends any length along the individual wire core/insulation combinations sufficient to cover the bending node 220 and securely attach to the ends of the wire core/insulation combinations. In other implementations, another hinged structure (e.g., a metal hinge, spring-loaded hinge, etc.) is used in conjunction with or in lieu of the flexible tube 230.

The wire core/insulation/flexible tube combination is mostly covered in a padded tube 232 that covers the bending node 220 and a majority of the remaining length of the wire core/insulation/flexible tube combination. As a result, the padded tube 232 covers the portion of the clamp 202 intended to come in contact with the user's penis. The padded tube 232 may be loosely or securely attached to the wire core/insulation/flexible tube combination and increases the overall diameter of the clamp 202 to a level sufficient to provide comfort to the user, but still sufficiently small to be effective and not overly bulky in size. The padded tube 232 may stop short of covering the entire length of the clamp 202 so that the coated ends 222, 224 are exposed, allowing them to be used as hooks (see FIG. 3). In other implementations, separate hook structures are attached to the coated ends 222, 224.

The padded tube 232 is resilient, enabling the clamp 202 to readily bend at the bending node 220 without failure. More specifically, the flexible tube 230 may be made of rubber (natural or synthetic) foam or various textile materials. In various implementations, the padded tube 232 is made of a non-absorbent material to aid the user in maintaining the clamp 202 in a sanitary condition (i.e., making the clamp 202 easy to clean). In other implementations, the padded tube 232 is made of an absorbent material that can absorb blood, sweat, urine, or other bodily fluids and wick them away from the user's penis. Further, the padded tube 232 may be a variety of shapes (e.g., round or flat) and have a variety of surface textures (smooth, patterned, etc.).

In some implementations, the padded tube 232 is permanently affixed to the clamp 202 and the clamp 202 is replaced periodically. In other implementations, the padded tube 232 is made of a disposable material that is periodically removed and replaced by the user. For example, the padded tube 232 may have a seam (not shown) along its length that is selectively secured (e.g., via hook-and-loop tape or other selective fastener) to form the padded tube 232 around the wire core/insulation/flexible tube combination. The seam may then be oriented opposite a surface of the padded tube 232 that engages with the user's penis to prevent irritation caused by contact with the seam. The padded tube 232 may be replaced once the underlying material loses its resilience or to maintain a desired sanitary condition of the clamp 202. The padded tube 232 may have medication or lubricant embedded therein or coated thereon depending on the user's requirements in treating a specific medical condition or providing a comfortable fit. The padded tube 232 may also be made of an anti-microbial/bacterial/fungal substance or coated with the anti-microbial/bacterial/fungal substance to maintain the padded tube 232 in a sanitary condition and perhaps treat the user's specific medical condition (e.g., treating fungus or skin sores). Further yet, the padded tube 232 may include reactive reagents that upon application of urine, sweat, blood, or other bodily fluids provide evidence of the user's medical status to the user or the user's physician (e.g., blood sugar level, blood in urine, etc.).

FIG. 3 is a cross-section of an example user conformable incontinence clamp 302 in a closed position. The clamp 302 includes solid wire cores 316, 318 that generally run through the center of the clamp 302. The wire core 316 is separated from the wire core 318 at a bending node 320 of the clamp 302 and the solid wire cores 316, 318 include coated ends 322, 324 opposing the bending node 320. Wire core 316 is covered in insulation 326 and wire core 318 is covered in insulation 328. The wire core/insulation combinations are partially covered with a flexible tube 330, at least covering the bending node 320. The wire core/insulation/flexible tube combination is mostly covered in a padded tube 332 that covers the bending node 320 and a majority of the remaining length of the wire core/insulation/flexible tube combination.

In the depicted closed position, the coated ends 322, 324 of the solid wire cores 316, 318 opposing the bending node 320 are shown bent and partially hooked around one another to maintain the closed position. Further, the direction and magnitude that each solid wire core 316, 318 is bent may affect how the coated ends 322, 324 hook together. The user may vary the direction and magnitude that each solid wire cores 316, 318 is bent to achieve a desired fastening of the coated ends 322, 324 of the solid wire cores 316, 318 together. In other implementations, the clamp 302 incorporates other selective attachment mechanisms (e.g., hooks, snaps, magnets, buttons, etc.) at the ends of the solid wire cores 316, 318 opposing the bending node 320.

Overall dimensions of the clamp 302 may vary widely depending on user requirements. In some implementations, the clamp 302 has a standard size intended to fit all users. In other implementations, the clamp 302 comes in several discrete sizes that are selected based on an individual user's needs. For example, the clamp 302 may have a total outside length 334 ranging from 75 mm to 82 mm with a cross-sectional diameter 336 ranging from 10 mm to 13 mm. Further, the clamp 302 may have a total inside length 335 ranging from 50 mm to 75 mm.

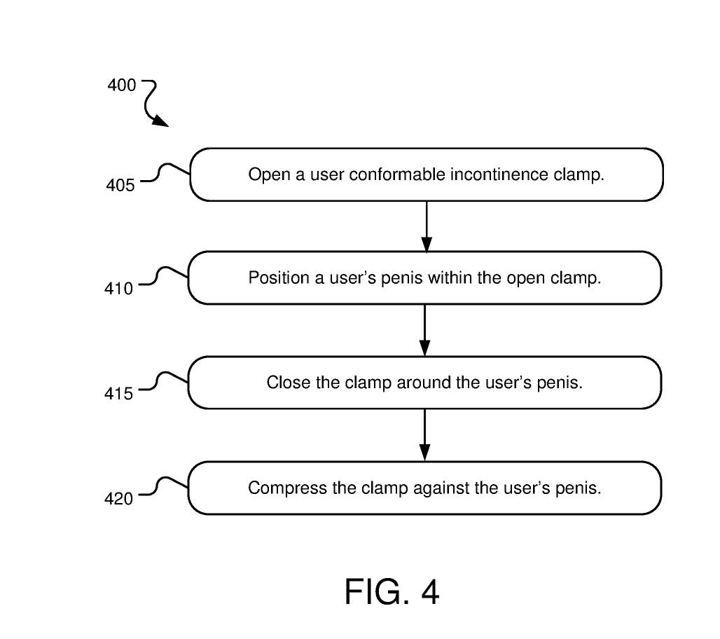

FIG. 4 illustrates example operations 400 for using a user conformable incontinence clamp. An opening operation 405 opens the user conformable incontinence clamp. More specifically, a user may detach a first selective attachment mechanism (e.g., a first coated portion, flattened portion, and/or hook) attached at a first end of the clamp from a second selective attachment mechanism (e.g., a second coated portion, flattened portion, and/or hook) attached to a second end of the clamp. The user may then elastically open the clamp by bending the clamp at a bending node, which separates the first end from the second end of the clamp. A positioning operation 410 positions the user's penis within the open clamp. More specifically, the user may manually place the clamp around his penis in a particular area that the user intends to clamp.

A closing operation 415 closes the clamp around the user's penis. The user may elastically close the clamp by bending the clamp at the bending node, which brings the first and second ends of the clamp back in close proximity to one another. The user re-attaches the first and second selective attachment mechanisms located at the ends of the clamp to one another to form a continuous loop around the user's penis. Further, the closing operation 415 may be achieved solely with the user's hands, and in some cases only one of the user's hands. Still further, the opening operation 405, the positioning operation 410, and the closing operation 415 may be repeated with or without readjusting the clamp (see compression operation 420) so that the user may quickly open the clamp, urinate, and close the clamp without having to significantly readjust the clamp.

A compression operation 420 compresses the clamp against the user's penis. More specifically, the user applies compressive force on the clamp to plastically conform the clamp to a desired shape and level of compression on the user's penis to provide a desired level of UI protection and comfort for the user. Further, the compression operation 420 may be periodically repeated to adjust the compression on the user's penis to maintain the desired level of UI protection and comfort for the user.

In one example implementation, the user bends a lower portion of the clamp upward, which causes a hook at an end of the lower portion to spread outward. The user may bend the hook at the end of the lower portion inward to maintain attachment with a corresponding hook at an end of an upper portion of the clamp. To loosen the clamp, the user may bend the upper portion of the clamp upward and perhaps adjust one or both of the hooks. The lower portion of the clamp may then be straightened a bit to adjust the clamp to the user's comfort level. Finally, the upper portion may be bent again to achieve the desired level of protection and comfort to the user. These steps may be repeated iteratively and in various orders to achieve the intended performance of the clamp.

The logical operations making up the embodiments of the invention described herein may be referred to variously as operations, steps, objects, or modules. Furthermore, it should be understood that logical operations may be performed in any order, unless explicitly claimed otherwise or the claim language inherently necessitates a specific order.

While the systems and methods disclosed herein are specifically directed at preventing or reducing the symptoms of UI, the systems and methods disclosed herein could be used to clamp other human body parts (or other mammal body parts) for the purposes or reducing or preventing the flow of fluid there through. For example, the systems and methods disclosed herein may be used in a triage situation to prevent or reduce blood flow through an injured body part (e.g., a penis, an arm, a finger, a leg, a tongue, etc.) decreasing the injured individual's blood loss as a result of the injury. Still further, the systems and methods disclosed herein may be used to perform the function of a pinch valve in a variety of systems (e.g., pinching closed medical fluid tubes).

The above specification, examples, and data provide a complete description of the structure and use of exemplary embodiments of the invention. Since many embodiments of the invention can be made without departing from the spirit and scope of the invention, the invention resides in the claims hereinafter appended. Furthermore, structural features of the different embodiments may be combined in yet another embodiment without departing from the recited claims.

What is claimed is:

1. A user conformable incontinence clamp comprising:
a first wire including a first selective attachment mechanism on a first end of the first wire;
a second wire including a second selective attachment mechanism on a first end of the second wire;
a flexible hinge connecting a second end of the first wire to a second end of the second wire; and
a padded tube encompassing a majority of the clamp, wherein the first selective attachment mechanism is configured to selectively attach to the second selective attachment mechanism to close the clamp around a user's penis, and wherein the first wire and the second wire are selectively conformable to the user's penis.

2. The user conformable incontinence clamp of claim 1, further comprising:
a first insulation section fixedly attached to and encompassing a majority of the first wire; and
a second insulation section fixedly attached to and encompassing a majority of the second wire.

3. The user conformable incontinence clamp of claim 1, wherein the first selective attachment mechanism and the second selective attachment mechanism each comprise one or more of a hook, a flattened wire end, and a coated wire end.

4. The user conformable incontinence clamp of claim 1, wherein the second end of the first wire and the second end of the second wire are axially aligned within the flexible hinge.

5. The user conformable incontinence clamp of claim 4, further comprising:
a gap between the axially aligned ends of the first wire and the second wire.

6. The user conformable incontinence clamp of claim 1 having a total outside length ranging from 75 mm to 82 mm and a cross-sectional diameter ranging from 10 mm to 13 mm.

7. The user conformable incontinence clamp of claim 1, wherein the flexible hinge is elastically deformable and the first wire and the second wire are each plastically deformable.

8. The user conformable incontinence clamp of claim 1, wherein the first wire and the second wire are each metallic and the flexible hinge is rubber.

9. The user conformable incontinence clamp of claim 1, wherein the padded tube includes one or more of a disposable material, a non-absorbent material, a foam, a textile material, an embedded medication, a medication coated thereon, an embedded lubricant, a lubricant coated thereon, an embedded anti-microbial/bacterial/fungal substance, an anti-microbial/bacterial/fungal substance coated thereon, an embedded reactive reagent substance, a reactive reagent substance coated thereon.

10. A method of using a user conformable incontinence clamp comprising:
positioning the user conformable incontinence clamp at a desired location on a user's penis;
closing the clamp around the user's penis; and
compressing the clamp to conform to a desired shape and level of compression on the user's penis.

11. The method of claim 10, further comprising:
opening the user conformable incontinence clamp prior to the positioning operation.

12. The method of claim 11, wherein the opening operation includes:
selectively detaching a first selective attachment mechanism from a second selective attachment mechanism; and
elastically deforming a flexible hinge to separate the first selective attachment mechanism from the second selective attachment mechanism.

13. The method of claim 10, wherein the closing operation includes:
elastically deforming a flexible hinge to bring a first selective attachment mechanism in close proximity to a second selective attachment mechanism; and
selectively attaching the first selective attachment mechanism to the second selective attachment mechanism to form a continuous loop.

14. The method of claim 10, wherein a padded tube of the conformable incontinence clamp is placed in contact with the user's penis during the closing operation.

15. The method of claim 10, wherein the compressing operation includes plastically deforming one or both of a first wire and a second wire within the user conformable incontinence clamp to conform to the desired shape and level of compression on the user's penis.

16. The method of claim 10, wherein the compressing operation further includes plastically deforming a first wire to a concave shape intended to cradle a top surface of the user's penis.

17. The method of claim 10, wherein the compressing operation further includes plastically deforming a second wire to a convex shape intended to apply pressure on the user's urethra via a bottom surface of the user's penis.

18. The method of claim 10, wherein the compression operation achieves one or both of a desired level of involuntary urination protection and a desired level of comfort to the user.

19. The method of claim 10, wherein the compressing operation is performed prior to the positioning operation.

20. A user conformable incontinence clamp comprising:
a first wire including a coated wire end;
a first insulation section fixedly attached to and encompassing a majority of the first wire;
a second wire including a coated wire end;
a second insulation section fixedly attached to and encompassing a majority of the second wire;
a flexible hinge connecting an end of the first wire opposing the coated wire end to an end of the second wire opposing the coated wire end; and
a padded tube encompassing a majority of the clamp, wherein the first selective attachment mechanism is configured to selectively attach to the second selective attachment mechanism to close the clamp around a user's penis, and wherein the first wire and the second wire are selectively conformable to the user's penis.
